(19) United States
(12) Patent Application Publication
(10) Pub. No.: US 2011/0218166 A1
(43) Pub. Date: Sep. 8, 2011

(54) MUCOADHERENTS COMPOSITIONS AND THEIR USE

(75) Inventors: Haline Fernanda Santana Castanho, Sao Paulo City (BR); Lupércio Calefe, Sao Paulo City (BR)

(73) Assignee: Incrementha PD&I - Pesquisa, Desenvolvimento e Inovecao de Famacos e Medicamentos Ltda., Sao Paulo (BR)

(21) Appl. No.: 13/059,032

(22) PCT Filed: Aug. 14, 2009
(86) PCT No.: PCT/BR2009/000255

§ 371 Date: May 25, 2011

(30) Foreign Application Priority Data
Aug. 14, 2008 (BR) PI0803568-7

(51) Int. Cl. A61K 31/7048; A61K 47/32; A61K 31/56; A61K 31/4025; A61K 31/43; A61K 31/545; A61K 31/65; A61K 31/7036; A61K 31/496; A61K 31/4164; A61K 31/4196; A61P 15/02; A61P 31/04; A61P 31/10; A61P 33/02

(52) U.S. Cl. 514/29; 514/772.1; 514/182; 514/422; 514/192; 514/200; 514/152; 514/36; 514/37; 514/254.07; 514/399; 514/398; 514/383

(57) ABSTRACT

The present invention has as its objective to provide mucoadherent compositions with enhanced properties of bioadhesivity, consistency, stability and vaginal pH regulation. It can also be the carrier of an active principle for the treatment or prophylaxis of disturbances or diseases caused in mucosa, particularly in the vaginal tract, as well as their use.

### FIELD OF THE INVENTION

The present invention relates to a mucoadherent pharmaceutical composition, substantially transparent, appropriate for use as a pH regulator vaginal formulation and also to serve as carrier vehicle of an active principle for the treatment of microbial diseases or disturbances caused in mucosa, particularly in the vaginal mucosa.

### BACKGROUND OF THE INVENTION

The vaginal mucosa is an appropriate environment for the survival of microorganisms. These microorganisms are responsible for maintaining the vaginal pH acidic around 2.0 to 4.5 by inhibiting the growth of opportunist pathogens and by promoting resistance to infections of pathogenic microorganisms. This way, any alteration in the normal vaginal flora or in the pH can cause a series of disturbances in the vaginal mucosa, including diseases caused by microbial infections. The equilibrium of the vaginal ecosystem is maintained by complex interactions between the said normal vaginal flora, the microbial metabolism products, the hormonal state and the immune response of the host.

The vagina is occupied by numerous bacteria of different species which are considered commensal (normal flora), but that can, in special situations, become pathogenic. The Doderlein's bacilli are the predominant microorganism in the vaginal media, representing 90 to 95% of the microorganisms present in the normal flora. The commensal microorganisms are responsible for maintaining the acidic vaginal pH (2.0 to 4.5) and consequently, inhibiting the growth of several other bacteria that are potentially harmful to the vaginal mucosa.

However, many factors can cause alterations in the vaginal ecosystem resulting in drying, pH alteration and disturbances in the vaginal flora, symptoms which are many times observed in women in the postmenopausal period.

During menopause, due to the reduction in the production of some hormones, the woman presents low vaginal lubrication. The lack of estrogen, observed in women during menopause, causes urogenital alterations, which lead to atrophy of the vaginal epithelium, making the tissue fragile to the point of bleeding. In the vagina, the atrophy causes the narrowing and shortening, loss of elasticity and reduction of secretions, causing the vaginal dryness. When the vagina becomes dry, the friction of the penis during sexual relations can hurt it, besides being able to cause vulvovaginitis.

The low concentration of estrogen is also one of the causes of modifications in the vaginal flora, which can result in an alteration of the vaginal pH and facilitate the appearance of an unspecific flora that predisposes the mucosa for the occurrence of vaginitis.

In order to improve the vaginal ecosystem, especially in women during menopause, it is advisable the use of moistening and/or acidifying creams, as well as the possibility of hormonal reposition.

Many formulations for vaginal use has been proposed, always in the sense of solving problems associated by the state of the art, related to: (i) provide a vehicle of controlled release of the active principle in order to meet the needs of a rapid release, prolonged release, or both, according to the disturbance or the disease to be treated; (ii) consistency of the product to be administered; (iii) the equilibrium between the hydrophilic and hydrophobic profile of the product in order to guarantee the bioavailability of the active principle in the vaginal environment; (iv) appropriate bioadhesivity of the product to the vaginal mucosa and (v) factors that cause allergic reactions or irritability of the mucosa.

The compliance of all these characteristics simultaneously constitutes a non-trivial task, especially because the vaginal formulations need to be non-toxic and non-propitious to the growth of microorganisms that cause vaginitis and other disturbances of the vaginal mucosa. Therefore, in the state of the art there is a large number of patent documents with focus on mucoadherent formulations for vaginal use, with the objective of, mainly, the improvement of vaginal moisture, the maintenance of a healthy pH and principally the delivery of active principles.

In the world market there is a great variety of acidifying and/or moistening (or humectant) vaginal products with the function of improve the vaginal ecosystem. Among the commercialized products it can be highlighted the KY gel Brand® (Johnson & Johnson), the Replens® (Columbia Laboratories) and the RepHresh® (Columbia Laboratories).

Among the products described in the state of the art it is worth highlighting the ones mentioned in the patent PI 9007807-1, corresponding to the patents EP 431,719, U.S. Pat. No. 6,017,521, U.S. Pat. No. 5,968,500 and U.S. Pat. No. 5,474,768 (Columbia Laboratories). Such products comprise a bioadhesive polymer (for example, polycarbophil, Carbopol®, among others) and alternatively an enhancer of consistency (for example, Carbopol®, carboxymethylcellulose, hydroxypropilcellulose, among others), see U.S. Pat. No. 5,968,500 and U.S. Pat. No. 6,017,521, being important to highlight that in the presented examples of the patent document PI 9007807-1 and its correspondent patents, the formulations always contain different proportions, among them, of bioadhesive polymer and consistency enhancer polymer. More than that, it is mentioned that “a greater amount of a consistency-enhancing is generally utilized with a smaller amount of bioadhesive polymer, and vice-versa. For example, a composition at a pH value of 2.2-2.5 containing 0.25 weight percent of polycarbophil as the bioadhesive requires about 8-10 weight percent CARBOPOL® 934 to achieved a viscosity appropriate for mechanical placement in the vagina” (see U.S. Pat. No. 5,968,500, column 11, second paragraph). In the example 4 of the patent EP 431,719 (corresponding to the patent PI 9007807-1) it is provided a formulation containing polycarbophil (2%), Carbopol® 934 (1%), Myverol® (1%, dispersant agent), 50 ml of mineral oil, 100 ml of glycerin, methylparaben (0.1% preservative agent), deionized water (q.s.p.) and pH adjustment to 2.4 with sodium citrate in HCl. It is important to observe that, according to the example 5 of this patent (EP 431,719), it is mentioned that a formulation containing 2% polycarbophil and 1% Carbopol® 934 (as the one described in the example 4) presents an appropriate viscosity, while compositions containing 1% Carbopol® 934 and 1% or 3% polycarbophil presented an inadequate viscosity because the former (1% Carbopol® 934 and 1% polycarbophil) was considered too “fine”, despite of its creamy consistency, and the latter (1% Carbopol® 934 and 3% polycarbophil) was too thick to apply.

In the patent U.S. Pat. No. 4,226,848 it is described a composition of controlled release comprising a polymeric matrix and an active principle in the matrix, the matrix comprising 50 to 95% of an cellulose ether (for example, hydroxypropilcellulose) and 50 to 5% of an acrylic homo- or copolymer (for example, Carbopol® 934). It is mentioned the fact that the formulation has as its objective to improve the bioadhesivity and avoids the irritability of the vaginal mucosa common in the previous products.

Many are the documents that describe compositions for the treatment and/or moistening of mucosa, including vaginal mucosa, containing polycarbophil (for example, Noveon-AA1®) and/or a carbomer (for example, Carbopol® 934P, Carbopol® 974P, Carbopol® 976P, and similar). Among such documents can be highlighted EP 719,146, WO 99/13862, US 2001/0031251 (U.S. Pat. No. 6,479,045) and PI 0213584-1 (corresponding to WO 03/037382) that present examples containing both polymers (polycarbophil and carbomer).

Bioadhesive polymers have as characteristics the insolubility in water associated to the capacity of water absorption. Due to these characteristics, such polymers have been used in systems of drug release of several via of administration, including intravaginal gels. When applied in the intravaginal form, the bioadhesive gels produce a moistening film over the vaginal tissue, which stays adhered to the surface of epithelial cells. The moistening action is due to the release of the water previously absorbed by the polymer and consequent hydration of the adjacent cells. The hydration of the epithelium lubricates the vaginal wall and reduces the occurrence of the symptoms associated to drying, such as itching, irritation and dyspareunia. Besides that, gels based on bioadhesive polymers can contribute to the reduction of the vaginal pH in the range of 2.0 to 4.5, which is the vaginal pH of healthy women pre-menopause and also is the ideal pH to avoid the development of vaginal infections.

The teachings of the document WO 2005/007194 are even more elucidative of the complexity of the appropriate compositions to meet all the exigencies of treatment and/or moistening of mucosa, especially the vaginal mucosa. In this document semi-solid mucoadhesive formulations are described comprising at least two bioadhesive polymers and one active ingredient, being the first polymer of the acrylic acid type (for example, Noveon-AA1®) and the second polymer being of the gelifying type (for example, Carbopol® 934P, Carbopol® 971P), the said formulations containing, also, a moistening/humectant agent (for example, glycerin), a fatty/lipophilic component (for example, paraffin, Vaseline, mineral oil) a solubilizing/emulsifying agent (Labrafil® M1944), a neutralizing agent for the adjustment of the pH between 2 and 6 and water. In the document WO 2005/007194 it is mentioned that the concentrations of the first and the second polymer varies from 0.1 to 5%, being preferred the ranges of 0.5 to 2.5% to the first polymer (polycarbophil) and of 0.1 to 1.0% to the second polymer (Carbopol®), being more preferred, still, the ranges of 0.75 to 1.5% and of 0.25 to 0.5% for the first and the second polymer, respectively. It is interesting to observe that in the examples A to H, K and 2 to 11 (formulations of progesterone (examples 2 to 6), of estriol (examples 1, 7 and 8), of clotrimazole (examples 9 and 10) and of clindamycin (example 11)) the ration of Carbopol®/polycarbophil is of 1:3 (0.5% Carbopol® and 1.5% polycarbophil in the formulations of the examples A to H, K and 7 and 8; and 0.25% Carbopol® and 0.75% polycarbophil in the formulations in the examples 2 to 6 and 9 to 11) and of 1:2 in the examples J, Q, P and R (0.5% Carbopol® and 1.0% polycarbophil).

Although the formulations described in the document WO 2005/007194 have been representing an advance regarding improvement of the consistency of composition for moistening and treatment of mucosa, it was verified that in the case of compositions for vaginal use, such compositions do not meet the exigencies of consistency due to peculiarities of the vaginal administration of a product that needs to present more adherence to avoid draining and more comfort to avoid the feeling of a hydrophobic product on contact to the mucosa.

In summary, despite of the intense studies that resulted in the several known mucoadhesive formulations, it was verified that the described products in the state of the art do not fully meet the exigencies of a product for vaginal administration, especially those related to a sufficient bioadhesivity to the mucosa, appropriate viscosity/consistency to avoid product draining, an humectant sensation without the discomfort caused by the contact to oily products, presenting low or no irritability of the mucosa that can be caused by the formulation, satisfactory organoleptic properties, appropriate pH to aid the maintenance of the normal vaginal flora and prevention of the development of pathogens. The compliance of all of these exigencies is the purpose of the compositions of the present invention.

### SUMMARY OF THE INVENTION

The present invention has as its objective to provide mucoadherent compositions with enhanced properties of bioadhesivity, consistency, stability, moistening and vaginal pH regulation. It can also be the carrier of an active principle for the treatment or prophylaxis of disturbances or diseases in the vaginal tract.

A first embodiment relates to a mucoadherent composition, essentially free of oily substances comprising: (a) 0.25 to 1.5% of a bioadherent polymer, preferentially 0.5 to 1.0%; (b) 0.25 to 1.5% of a gelifying polymer, preferentially 0.5 to 1.0%; (c) 17 to 25% of pharmaceutically acceptable excipients and (d) water, with the condition of the bioadherent polymer/gelifying polymer ratio be 1:1. The said composition presents itself preferentially in the vaginal pharmaceutical form. Preferentially, the composition is in the form of an aqueous gel. Particularly, comprises around 25% to 90% of water. Still more preferentially, the composition comprises at least around 70% of water.

A second embodiment of the invention is regarding a mucoadherent composition, essentially free of oily substances, carrier of an active principle for the treatment or prophylaxis of vaginal disturbances or diseases comprising: (a) a therapeutically efficient quantity of a selected active principle from group consisting of hormonal, antibacterial, antifungal, antiprotozoan, antiviral, spermicidal agents, local anesthetic, anti-inflammatory and anti-spasmodic and (b) an aqueous-based formulation comprising (i) 0.25 to 1.5% of an bioadherent polymer, preferentially 0.5 to 1.0%; (ii) 0.25 to 1.5% of a gelifying polymer, preferentially 0.5 to 1.0%; (iii) 17 to 25% of excipients and (iv) water, with the condition of the bioadherent polymer/gelifying polymer ratio be 1:1. Preferentially, the composition is in the form of an aqueous gel. Particularly, the composition comprises around 25% to 90% of water. Still more preferentially, the composition comprises at least around 70% of water.

A third embodiment of the invention is regarding the use of mucoadherent compositions, as described above, that have as their objective the vaginal pH regulation, particularly to a value of 2.0 to 4.5, as well as a vaginal pharmaceutical form and preparation of the said pharmaceutical form for the treatment or prophylaxis of disturbances or diseases of the vaginal tract.

### DETAILED DESCRIPTION OF THE INVENTION

The composition of the present invention is directed, in a first embodiment, to the regulation of the vaginal mucosa pH, particularly to a pH value in the range of 2.0 to 4.5, being this pH range responsible for the maintenance of the flora and for the inhibition of growth of pathogenic microorganism that cause vaginal disturbances and diseases.

The invention is based on the verification that an adequate formulation, essentially free of oily substances, for vaginal administration comprises a first polymer to confer bioadhesivity of the product to the walls of the vaginal mucosa and a second polymer to confer gelifying characteristics to the product, said first and second polymers being in low concentrations in the aqueous formulation and in the first/second polymer ratio of 1:1.

The first polymer with bioadhesivity property can be selected among the bioadhesive polymers mentioned in the patents U.S. Pat. No. 5,968,500 and U.S. Pat. No. 6,017,521, herein incorporated in their entirety, being particularly preferred the polycarbophil, such as the acid polycarbophil from the brand Noveon-AA1®.

The second polymer with gelifying characteristics can be selected among the gelifying or matrix producer agents mentioned in the document WO 01/066084, herein incorporated in its entirety, or consistency enhancers agents cited in the U.S. Pat. No. 6,017,521, herein incorporated in its entirety, or still from the group of carbomer polymers of the Carbopol® series, including Carbopol® 934P, Carbopol® 971P, Carbopol® 974P, Carbopol® 976P, Preferentially, the second polymer with gelifying characteristics is selected from the group consisting of Carbopol® 934P, Carbopol® 971P, Carbopol® 974P, Carbopol® 976P and still more preferentially, the second polymer with gelifying characteristics is the Carbopol® 974P.

The composition of the present invention is of aqueous-based type and contains a pH regulator agent with the intent of maintaining the pH of the formulation in the range of 3.5 to 5.0, and still more preferentially to a value in the range of 4.1 to 4.5, selected from the group consisting of lactic acid, citric acid, tartaric acid, benzoic acid, alginic acid, sorbic acid, diaminotetracetic acid (EDTA), acetic acid, malic acid and triethanolamine, as well as their respective salts and mixtures thereof, still more preferentially the pH regulator agent is selected among lactic acid, sorbic acid and triethanolamine, being the most preferred the triethanolamine.

Additionally, the mucoadherent composition of the present invention contain one or more excipients or adjuvants selected among lubricants, plastifying agents, preservative agents, colorants, flavoring agents and moistening (humectant) agents that can be combined based on the knowledge of an specialist in pharmaceutical formulations technique.

The moistening (humectant) agent can be selected from the group consisting of polyetheleneglycol, propilenoglycol, sorbitol, triacetine and glycerin, being the most preferred the glycerin.

The preservative agent can be selected from the group consisting of benzoic acid, sodium benzoate, benzalconic cloride, phenylmercury nitrate, chlorexidine, parabens and is sorbic acid, being the most preferred the sorbic acid.

According to a general aspect, the compositions comprised in the present invention are essentially free of oily substances. It is understood as oily substances those with hydrophobic profile and substantially immiscible in water, such as, for example: mineral oil, triglycerides, fatty acids, hydrogenated vegetable oil, and similar. The term “essentially free of oily substances” can be understood as comprising up to 2% of the said oily substances.

According to a second general aspect, the compositions comprised in the present invention are essentially free of irritating substances, such as ethanol, parabens, among others.

The second embodiment of the present invention relates to a mucoadherent composition, essentially free of oily substances, carrier of an active principle for the treatment or prophylaxis of vaginal disturbances or diseases comprising: (a) a therapeutically efficient quantity of a selected active principle from group consisting of hormonal, antibacterial, antifungal, antiprotozoan, antiviral, spermicidal agents, local anesthetic, anti-inflammatory and anti-spasmodic and (b) a formulation base corresponding to the composition described above.

The active principle of the mucoadherent composition according to the present invention can be: (i) from the group of hormones, such as estrogens, for example, estriol and 17-β-estradiol or progestogens, for example, progesterone and medrogestone; (ii) from the group of antibacterial, such as clindamycin, penicillin, cefalosporin, tetracyclin, gentamycin, erythromycin, kanamycin, streptomycin, among others; (iii) from the group of antifungal, such as miconazole, itraconazole, fluconazole, ketoconazole and others; (iv) from the group of antiprotozoan, such as tinidazole, metronidazole and others; (v) from the group of antiviral, such as the anti-HIV agents or the anti-herpes agents; (vi) from the group of the spermicidal, such as nonoxynol-9, menphegol; (vii) from the group of the local anesthetic, such as lidocaine and its isomers, benzocaine, procaine; (viii) from the group of anti-inflammatory, such as the corticosteroids and the non-steroids and (ix) from the group of anti-spasmodic, such as terbutaline, salambutol, hexoprenaline and others.

The third embodiment of the invention relates to the use of the mucoadherent composition of the invention in the vaginal moistening and pH regulation to a value in the range of 2.0 to 4.5. The bioadhesive action, which is conferred by the composition of the invention comprising a bioadherent polymer combined to a gelifying agent that increases significantly the adherence of the product to the walls of the vaginal mucosa, avoids the detachment of amines a enables the restoration of the of the Doderlein's bacilli acidophilus as dominant component of the flora and making the vaginal environment hostile to the undesired proliferation of other microorganisms. The mucoadherent composition of the present invention presents moistening action of the vaginal channel reducing, therefore, the consequences of vaginal dryness that occurs naturally in the postmenopausal period. The bioadhesive polymers, used in the composition of the present invention, have as their characteristics the insolubility in water associated with the capacity of water absorption. When applied in the intra-vaginal form, the bioadhesive gels produce a humidifying film over the vaginal tissue, which stays adhered to the surface of the epithelial cells. The moistening action is due to the release of the water previously absorbed by the polymer and consequent hydration of the adjacent cells. The hydration of the epithelium lubricates the vaginal wall and reduces the occurrence of the symptoms associated to drying, such as itching, irritation and dyspareunia.

Another important factor in the prevention or treatment of disturbances or diseases of the vaginal tract is the pH maintenance in the physiological range. The gels based on bioadhesive polymers can contribute to the reduction of the vaginal pH in the range of 2.0 to 4.5, which is the vaginal pH of healthy women pre-menopause and also is the ideal pH to avoid the development of vaginal infections. Therefore, the composition of the present invention is capable of maintaining the vaginal pH in the ideal range.

The fourth embodiment of the invention relates to the use of the mucoadherent composition of the invention as an aqueous-based carrier of an active principle for the treatment or prophylaxis of vaginal disturbances and diseases. The improved characteristics of the composition of the invention, such as, enhanced bioadhesivity and consistency, and the fact of being essentially free of substances that can cause irritability of the vaginal mucosa and of hydrophobic substances confer optimal properties to the mucoadherent composition of the invention in order to enable it to function as carrier of the active principle, promoting a higher bioavailability and permanence of it in the vaginal channel.

It must be understood that the examples and embodiments described herein are solely by way of illustration and that several modifications or changes, without departing from the spirit and scope thereof, in the light of themselves, will be apparent to those skilled in the art and must be included in the scope and spirit of this description and attached claims.

### EXAMPLES

The following experimental examples illustrate the present invention, without, however, limiting the coverage of its scope.

### Example 1

### Method of Preparation of the Formulations According to the Invention

In a beacker, provided with an agitation system, it is added deionized water and sorbic acid and maintained under agitation until complete homogenization.

Following, it is slowly dispensed over water and with the aid of a screen, under strong agitation, the polycarbophil (Noveon-AA1®—USP) and the Carbopol® 974P, agitating until the mixture becomes a translucent liquid. Following, it is reduced the agitation speed and the mixture is maintained for 20 minutes under these conditions.

After completion of the mixture phase, it is added glycerin and maintained the agitation until complete homogenization.

Lastly, the pH is verified and, if necessary, the pH correction is obtained with triethanolamine or 50% citric acid solution until the mixture reaches o pH value of 4.3. The pH value is important for gel adherence and to adjust the maintenance and/or correction of the vaginal pH.

Using the method described above, several formulations were prepared as defined as follows.

Three formulation (A to C) were prepared according to the invention, with different concentrations of the polymers (acid polycarbophil (Noveon-AA1®) and polyacrylic acid (Carbopol® 974P)) being used, maintaining the 1:1 ratio, while the concentrations of the other components of the formulations were maintained constant.

TABLE 1
Formulation A

| | Function in the | |
|---|---|---|
| Component | Composition | Quantity (%) |
| Polyacrylic acid | Gelifying polymer | 0.5 |
| (Carbopol ® 974P) | | |
| Acidic polycarbophil | Bioadhesive polymer | 0.5 |
| (Noveon-AA1 ® - USP) | | |
| Glycerin | Moistening agent | 20.00 |
| Sorbic acid | Preservative agent | 0.10 |
| Triethanolamine | pH regulator agent | q.s. pH |
| | | adjustment at |
| | | 4.3 ± 0.2 |
| Water (q.s.p.) | Vehicle | q.s.p. |

TABLE 2
Formulation B

| | Function in the | |
|---|---|---|
| Component | Composition | Quantity (%) |
| Polyacrylic acid | Gelifying polymer | 0.75 |
| (Carbopol ® 974P) | | |
| Acidic polycarbophil | Bioadhesive polymer | 0.75 |
| (Noveon-AA1 ® - USP) | | |
| Glycerin | Moistening agent | 20.00 |
| Sorbic acid | Preservative agent | 0.10 |
| Triethanolamine | pH regulator agent | q.s. pH |
| | | adjustment at |
| | | 4.3 ± 0.2 |
| Water (q.s.p.) | Vehicle | q.s.p. |

TABLE 3
Formulation C

| | Function in the | |
|---|---|---|
| Component | Composition | Quantity (%) |
| Polyacrylic acid | Gelifying polymer | 1.0 |
| (Carbopol ® 974P) | | |
| Acidic polycarbophil | Bioadhesive polymer | 1.0 |
| (Noveon-AA1 ® - USP) | | |
| Glycerin | Moistening agent | 20.00 |
| Sorbic acid | Preservative agent | 0.10 |
| Triethanolamine | pH regulator agent | q.s. pH |
| | | adjustment at |
| | | 4.3 ± 0.2 |
| Water (q.s.p.) | Vehicle | q.s.p. |

In the performed tests with the three formulations described above, it was verified that the Formulation C presented good consistency, good adherence and no draining, being a satisfactory formulation for administration in the vaginal channel. The formulation B, in which the concentration of the polymers was of 0.75%, presented optimal consistency, with good adherence and low draining.

It is important noting that, opposed to the teachings of the art, the compositions of the invention are based in low concentrations of polymers (polycarbophil and polyacrylic acid), and in which those are present in the 1:1 ratio, being this one of the main characteristic of the composition of the present invention and the reason for obtaintion of an optimal consistency of the aqueous composition of the invention.

### Example 2

### Determination of Viscosity of the Formulations According to the Invention

In order to determine the viscosity, it was used the viscosimeter of the brand Brookfield, model: DV-I (with Helipath dispositive), spindle S96, speed 6 rpm and temperature of 25° C. It is important to observe that alterations in any of these parameters can lead, as consequence, to the obtaintion of different results for equal compositions. Therefore, it only makes sense to compare viscosities of product submitted to tests in which the same parameters were applied.

TABLE 4
Results of the viscosity test

| Formulations | Polymers % | Viscosity (cPs) |
|---|---|---|
| A | 0.5 | 30,000 to 42,000 |
| B | 0.75 | 75,000 to 85,000 |
| C | 1.0 | 94,000 to 100,000 |

### Example 3

### Adherency Test (Test with Mucin) in the Formulations According to the Invention

In order to verify the mucoadhesivity of the product in the vaginal mucosa, an adherence test was performed with mucine.

For the execution of the test, the vaginal channel was simulated though the confection, with cellophane, of a channel with an aperture of 1 to 1.5 cm of diameter, 15 to 15.5 cm of length and 42 to 45° of inclination. In order to simulate the physiological mucous of the vaginal channel, it was added to the system simulator, a pork stomach's mucine-based preparation. After the preparation, the whole system was maintained at 37° C. for the execution of the tests.

The test was performed with three examples of formulation of the present invention; and with already commercialized products (commercial antifungal product 1 and commercial antifungal product 2) and (the KY gel Brand®—without an active principle, indicated for moistening the vaginal channel). The samples were added to the system through the use of a vaginal applicator in the quantities of 4 to 5 grams and maintained in the system for 2 hours. The result is described on Table 5.

TABLE 5
Results of the adherence test

| Product | Result |
|---|---|
| Formulation A | Approximately 300 mg of the |
| | product was drained |
| Formulation B* | Did not drain |
| Formulation C | Did not drain |
| KY gel brand ® | All the test sample was |
| | drained through the system |
| Commercial | All the test sample was |
| antifungal product 1 | drained through the system |
| Commercial | All the test sample was |
| antifungal product 2 | drained through the system |

*second test 50 mg of the product was drained

Therefore, the tests performed in the laboratory show that, when compared to other products available in the market, the formulations of the invention, principally the formulation B and C, remain for longer time on contact with the vaginal channel, without draining, while the products KY gel Brand®, and the two antifungal commercial products do drain. Besides causing discomfort to the user, the product draining also reduces the time and quantity of the product on contact with the mucosa. In the case of the two antifungal products, the time of contact with the mucosa surface is essentially important, once it contains active principles to treat vaginitis. Therefore, the formulations of the present invention, due to an adherence to the mucosa, are capable of maintaining the active principle for longer time on contact with the vaginal mucosa surface, which enables the use of a lower quantity of active principle with the same therapeutic effect.

The composition of the present invention can be indicated for lubrication, moistening and acidification of the vaginal pH, with good adherence and longer time of contact with the mucosa, allowing the relieve of the symptoms related to dryness and pH increase, symptoms observed principally in women in the postmenopausal period and the vaginal pH adjustment to a physiological value, therefore, avoiding the development of vaginal infections.

Other important characteristic of the composition of the invention are: (i) non-oily product; (ii) low irritability of the vaginal mucosa due to the fact that it is essentially free of substances that cause irritation to the mucous tissue, such as, for example, parabens and alcohol; (iii) the bioadhesive and gelifying polymers, used in the specified proportion, contribute to the reduction of the vaginal pH to the physiological value (2.0 to 4.5), being this the vaginal pH of pre-menopause healthy women, therefore, avoiding the development of vaginal infections; (iv) due to its aqueous type composition with specified proportions of the polymers, it enhances the lubrication characteristics, and the acidification of the vaginal pH.

All the publications and patent applications mentioned in the description are indicative of the level of those skilled in the art to which this invention relates to. All publications and patent applications are herein incorporated by reference to the same extent as each individual publication or each patent application were specifically and individually indicated to be incorporated by reference.

Despite of the invention has been described in some detailed by way of illustration and examples for a matter of clarity and understanding, it will be evident that certain changes and modifications can be practiced within the scope of the attached claims of this description.

1-40. (canceled)

41. A mucoadherent composition, wherein said composition is essentially free of oily substances and comprises:
(a) 0.25 to 1.5% of a bioadherent polymer;
(b) 0.25 to 1.5% of a gelifying polymer;
(c) 17 to 25% of pharmaceutically acceptable excipients; and
(d) water;
with the condition that the ratio of bioadherent polymer/gelifying polymer is 1:1.

42. The composition according to claim 41, wherein said composition comprises (a) 0.5 to 1.0% of a bioadherent polymer, (b) 0.5 to 1.0% of a gelifying polymer, and (c) up to 2% of oily substances.

43. The composition according to claim 41, wherein said composition is an aqueous gel.

44. The composition according to claim 41, wherein said composition contains 25% to 90% of water.

45. The composition according to claim 44, wherein said composition contain less than 70% of water.

46. The composition according to claim 41, wherein said bioadherent polymer is polycarbophil and the gelifying polymer is a polyacrylic polymer of the carbomer type.

47. The composition according to claim 46, wherein said polyacrylic polymer of the carbomer type is selected from the group consisting of Carbopol® 934P, Carbopol® 972P, Carbopol® 974P and Carbopol® 976P.

48. The composition according to claim 41, wherein said pharmaceutically acceptable excipients are selected from the group consisting of pH regulator agent, lubricant agent, plastifying agent, preservative agent, colorant, flavoring agent and moistening (humectant) agent.

49. The composition according to claim 48, wherein said pH regulator agent is selected from the group consisting of lactic acid, citric acid, tartaric acid, benzoic acid, alginic acid, sorbic acid, diaminotetracetic acid, acetic acid, malic acid, triethanolamine, as well as their respective salts and mixtures thereof.

50. The composition according to claim 48, wherein said moistening agent is selected from the group consisting of polyetheleneglycol, propilenoglycol, sorbitol, triacetine and glycerin.

51. The composition according to claim 48, wherein said preservative agent is selected from the group consisting of benzoic acid, sodium benzoate, benzalconic cloride, phenylmercury nitrate, chlorexidine and sorbic acid.

52. A mucoadherent composition, wherein said composition is a carrier of an active principle for treatment or prophylaxis of vaginal disturbances or diseases, essentially free of oily substances and comprising:
(a) a therapeutically effective quantity of an active principle selected from the group consisting of hormonal agents, antibacterial agents, antifungal agents, antiprotozoan agents, antiviral agents, spermicidal agents, local anesthetic agents, anti-inflammatory agents and anti-spasmodic agents; and
(b) an aqueous formulation base comprising:
(i) 0.25 to 1.5% of a bioadherent polymer; (ii) 0.25 to 1.5% of a gelifying polymer; (iii) 17 to 25% of pharmaceutically acceptable excipients; and
(iv) 25% to 90% of water,
with the condition that the bioadherent polymer/gelifying polymer ratio is 1:1.

53. The composition according to claim 52, wherein said composition comprises 0.5 to 1.0% of a bioadherent polymer, 0.5 to 1.0% of a gelifying polymer, and up to 2% of oily substances.

54. The composition according to claim 52, wherein said composition is an aqueous gel.

55. The composition according to claim 52, wherein said composition contain less than 70% of water.

56. The composition according to claim 52, wherein said active principle is a hormone selected from the group consisting of estrogens and progestogens.

57. The composition according to claim 52, wherein said antibacterial agent is selected from the group consisting of clindamycin, penicillin, cefalosporin, tetracyclin, gentamycin, erythromycin, kanamycin, streptomycin.

58. The composition according to claim 52, wherein said antifungal and/or antiprotozoan agent is selected from the group consisting of itraconazole, ketoconazole, miconazole, tinidazole, fluconazole, metronidazole agents or combinations thereof.

59. The composition according to claim 52, wherein said antiviral agent is selected from the group consisting of anti-HIV agent and anti-herpes agent.

60. The composition according to claim 52, wherein said pharmaceutically acceptable excipients are selected from the group consisting of moistening agent, preservative agent and pH regulator agent.

61. The composition according to claim 60, wherein said moistening agent is glycerin.

62. The composition according to claim 60, wherein said preservative agent is sorbic acid.

63. The composition according to claim 60, wherein said pH regulator agent is triethanolamine.

64. The composition according to claims 52, wherein said composition comprises 20% of glycerin, 0.1% of sorbic acid and triethanolamine in quantities needed for pH adjustment at 4.3±0.2 and water.

65. The composition according to claim 52, wherein said bioadherent polymer is polycarbophil and said gelifying polymer is of the carbomer type.

66. A method for the treatment of disturbances or diseases of the vaginal tract in a patient, comprising administering a mucoadherent composition as defined in claim 41.

67. A method for the treatment of disturbances or diseases of the vaginal tract in a patient, comprising administering a mucoadherent composition as defined in claim 52.
